(19) United States
(12) Patent Application Publication
(10) Pub. No.: US 2013/0217971 A1
(43) Pub. Date: Aug. 22, 2013

(54) INTUBATION GUIDE

(71) Applicant: Benje Boedeker, Omaha, NE (US)

(72) Inventor: Benje Boedeker, Omaha, NE (US)

(21) Appl. No.: 13/552,803

(22) Filed: Jul. 19, 2012

Related U.S. Application Data
(63) Continuation of application No. 12/079,031, filed on Mar. 24, 2008, now abandoned.
(60) Provisional application No. 60/896,619, filed on Mar. 23, 2007.

(51) Int. Cl. A61M 16/04; A61B 1/267

(52) U.S. Cl. CPC A61M 16/0488; A61B 1/267
USPC 600/188; 128/200.26; 600/194

(57) ABSTRACT

Apparatuses for guiding an endotracheal tube during intubation and associated methods of their use; the apparatuses formed to contain a plastic element able to hold deformations so as to conform the apparatus to the shape of a patient's endotracheal airway. The plastic element has sufficient give so as to minimize traumatic engagement with the inner surface of the airway lumen. Methods for using the apparatuses exploit indirect visualization where the person performing the intubation can shape the apparatuses so as to place the distal end into the field of view of the indirect visualization instrument even with minimal alignment of the patient's airway.

### CROSS REFERENCE TO RELATED APPLICATION

This application is a Continuation under 35 U.S.C. §120 of U.S. patent application Ser. No. 12/079,031 filed Mar. 24, 2008, entitled “INTUBATION GUIDE”, which claims the benefit under 35 U.S.C. §119 of U.S. Provisional Patent Application Ser. No. 60/896,619 filed Mar. 23, 2007, entitled “INTUBATION GUIDE, which applications are hereby incorporated by reference in their entirety.

### GOVERNMENT SUPPORT

This invention was made with government support under Grant No. W81xwh0610019 awarded by the Telemedicine and Advanced Technology Research Center. The government has certain rights in the invention.

### BACKGROUND

1. Field:

This invention relates to endoscopy and specifically to apparatuses and methods for improved visualization during endotracheal intubation.

2. Background:

A secure airway is essential to manage anesthetized or critically ill patients. Maintaining ventilation through an endotracheal tube is a critical to maintaining a patient's airway

In the current art, placing an endotracheal tube (endotracheal intubation or intubation), especially when done by those lacking extensive experience with the procedure, introduces substantial risk (See Field Airway Management Disasters 104 ANESTHESIA & ANALOGESIA 482 (2007) herein incorporated by reference). Accidental placement of the endotracheal tube in the esophagus, for example, can kill a patient if not immediately detected. In instances of a difficult airway, intubation may not be possible even when performed by skilled anesthesiologist.

The factors that make an intubation difficult complicate the person performing the inbutation (“the intubator”) ability to confirm the appropriate placement of the endotracheal tube. Confirmation of endotracheal tube placement is difficult given the anatomy of the patient's airway. In order to see the glottic opening, the ultimate destination of an endotracheal tube, the intubator must align the glottic opening with the mouth. Even after proper alignment multiple factors can obstruct visualization of the glottic opening resulting in a difficult airway.

The current art defines a difficult airway as the clinical situation where a clinically trained anesthesiologist experiences difficulty with face mask ventilation of the upper airway, difficulty with intubation, or both. Several factors can make for a difficult airway. Features of the patient, such as long upper incisors, a highly arched or narrow pallet, a low range of motion of the head and neck, or poor visibility of the uvula can indicate a difficult airway (See A clinical sign to predict difficult tracheal intubation; a prospective study 32 CANADIAN ANESTHETISTS' SOCIETY JOURNAL 429 (1985) herein incorporated by reference). The factors make for a difficult airway because they obstruct direct visualization of the glottic opening even when the intubator aligns the patient's airway.

The practice guidelines of the American Society of Anesthesiologists (See Practice Guidelines for Managing the Difficult Airway, 98 ANESTHESIOLOGY 1269 (2003) herein incorporated by reference) recognize multiple strategies to intubate a patient in the presence of a difficult airway. Utilizing a different laryngoscope blade, a laryngeal mask as an intubation conduit, fiberoptic intubation, a light wand, retrograde intubation or use of an intubating stylet, bougie or a tube changer are all alternative strategies where traditional laryngoscopy cannot intubate when a difficult airway is present.

The present art teaches that among the initial steps in endotracheal intubation is alignment of the patient's airway (See Orotracheal Intubation, 356 N. ENG. J. MED e15 (2007) herein incorporated by reference). Similarly, the associated tools used to guide an endotracheal tube are linear and unable to conform to the natural shape of the patient's airway are inoperable in the absence of an aligned airway. To that extent, the current art has yet to realize the potential of instruments that can indirectly visualize the glottic opening. Such indirect visualization instruments, like video laryngoscopes, video laryngeal masks or fiber optic bougies can visualize the glottic opening even when the patient's airway is unaligned (a process herein called “indirect visualization”). Current tools used to guide the placement of an endotracheal tube, however, cannot fully exploit the potential of indirect visualization because these tools can only be used in the presence of an aligned airway.

Indirect visualization instruments can see ‘around the corner’ and visualize the glottic opening even when an airway is difficult or incompletely aligned. The present art currently offers no tools that can similarly reach ‘around the corner’. As a result, the present art cannot realize the benefits of indirect visualization instruments because they cannot operate in the field of vision provided by an indirect visualization instrument. The present invention teaches an apparatus that can be used, in conjunction with indirect visualization, which will fulfill the enormous potential of indirect visualization and shift the current alignment paradigm.

### SUMMARY OF THE INVENTION

It is the object of the present invention to provide a new apparatus to guide the placement of an endotracheal tube.

It is the further object of the invention to guide the placement of an endotracheal tube by means of a plastic element allowing the intubator to shape an endotracheal intubation guide to substantially conform to the shape of a patient's airway.

It is the further object of the invention to utilize a shaped endotracheal intubation guide so as to exploit the view of the glottic opening provided by a Stortz DCI Video Laryngoscope or related indirect visualization instrument.

Accordingly, the present invention provides for an apparatus that can traverse the length of an endotracheal tube and comprises an element that conveys a degree of rigidity and the ability to undergo plastic deformation (a “plastic element”). Said plastic element having sufficient plasticity so as to retain a shape bent into it by an intubator during insertion into the patient's endotracheal airway but having sufficient give so as to minimize or avoid traumatic engagement with the inner surface of the airway lumen. The present invention teaches several embodiments of the plastic element: a ductile member received by the body of the endotracheal intubation guide, a characteristic of the material substantially comprising the endotracheal intubation guide or a member preformed to contain multiple bends conforming to the angles of a patient's unaligned airway.

The present invention further provides for the exploitation of the view of the glottic opening provided by indirect visualization instruments by teaching methods for utilizing the endotracheal intubation guide in conjunction with the indirect visualization instruments. Specifically, the present invention teaches methods for shaping, placing and confirming placement of the endotracheal intubation guide. The present invention further teaches methods for using the endotracheal intubation guide to place and confirm the placement of an endotracheal tube.

Additional objects, advantages and novel features of the present invention will be set forth in part in the description which follows, and in part will become apparent to those skilled in the art upon examination of the following, or may be learned from practice of the invention.

### BRIEF DESCRIPTION OF THE DRAWINGS

The invention will be better understood and other features and advantages will become apparent by reading the detailed description of the invention, taken together with the drawings, wherein:

FIG. 1A is an elevational view of the apparatus undergoing plastic deformation. FIG. 1B is a diagrammatic depiction of a formed apparatus useful in intubation.

FIG. 2A is an elevational view of one embodiment of the present invention undergoing plastic deformation. FIG. 2B cross sectional view of one embodiment and FIG. 2C is a longitudinal cross section of one embodiment.

FIG. 3A is a cutaway elevational view of an embodiment of the present invention. FIG. 3B is an elevational view of one embodiment of the present invention. FIG. 3C is an isometric view of one embodiment of the present invention with parts broken away to reveal details of construction.

FIG. 4A depicts a formed intubation guide and FIG. 4B depicts deformation in avoidance of traumatic engagement with the inner wall of the airway lumen.

FIG. 5A is an elevational view depicting a pre-formed embodiment of the invention undergoing deformation and FIG. 5B is an elevational view depicting the elasticity of the invention returning to substantially its original form.

FIG. 6A is a cross section of the patient's endotracheal airway showing the positioning of a visualization instrument and an intubation guide. FIG. 6B is a cross sectional view of the glottic opening and FIG. 6C is also a cross sectional view of the glottic opening.

### DETAILED DESCRIPTION

The following description is presented to enable any person skilled in the art to practice the present invention. Modifications to the embodiments described herein will necessarily be apparent to those skilled in the art. The present invention is not intended to be limited to the embodiments shown, but is to be accorded the widest scope consistent with the principles and features disclosed herein.

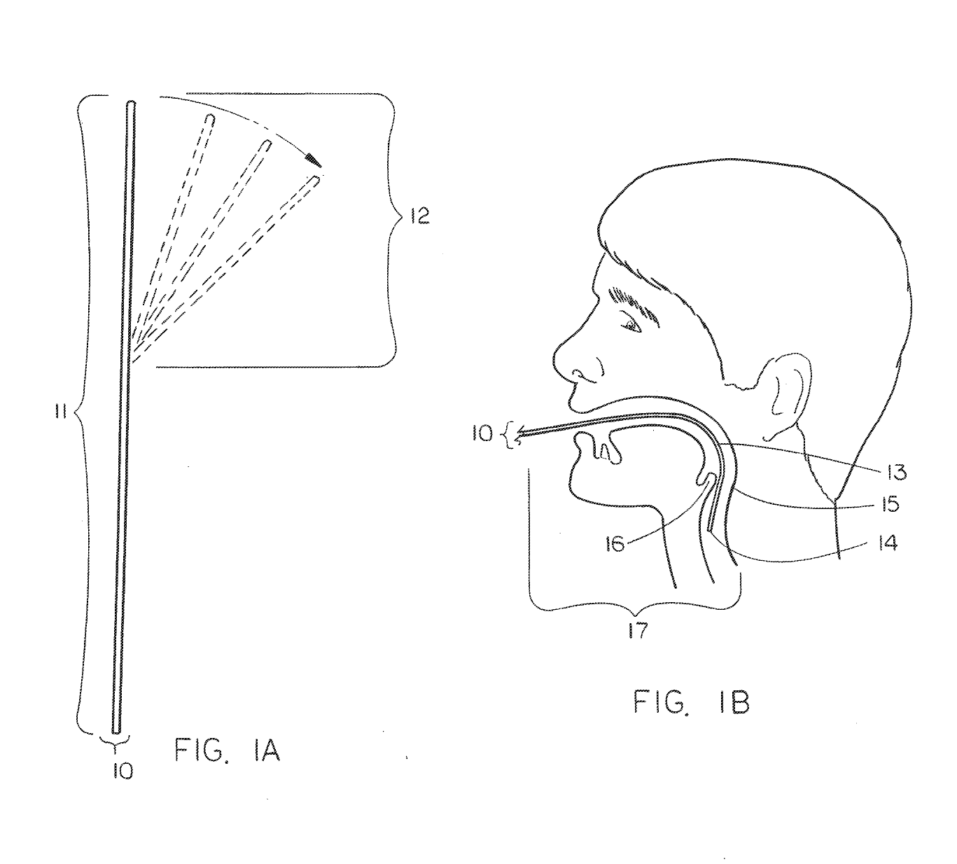

Looking at FIG. 1A, the present invention is an endotracheal intubation guide 10 that contains a plastic element 25 (not pictured) throughout all or substantially all of the body 11. The plastic element 25 is sufficiently malleable so as to enable the endotracheal intubation guide 10 to deform when bent 12 and sufficiently plastic so as to substantially maintain a shape bent 12 into it. The operator performing the intubation (“the intubator”) shapes the intubation guide 10 to substantially conform to the patient's endotracheal airway. The endotracheal intubation guide 10 and is formed to be a long, non-rigid, malleable member of sufficient length to traverse the length of the patient's endotracheal airway. To facilitate engagement with an endotracheal tube the member is generally round but could have an angular or other shape that can conform to the endotracheal tube lumen.

FIG. 1B depicts a shaped endotracheal intubation guide 13 that is better able to conform to the natural shape of a patient's endotracheal airway. When inserting the intubation guide into the patient's airway, the intubator inserts a first end into the patient's airway, the distal end 14. The shaped endotracheal intubation guide 13 is shaped so that the distal end 14 can be introduced into the airway 41, around the epiglottis 16, and into the glottic opening 62 (not pictured). When shaped properly, the shaped endotracheal intubation guide 13 can be used to place the distal end into the glottic opening 62 (not pictured) when the patient's airway is un-aligned 17. Critically, the endotracheal intubation guide 10 must have sufficient give so that the plastic element 25 (not pictured) will deform before traumatically engaging the walls of the lumen of the airway.

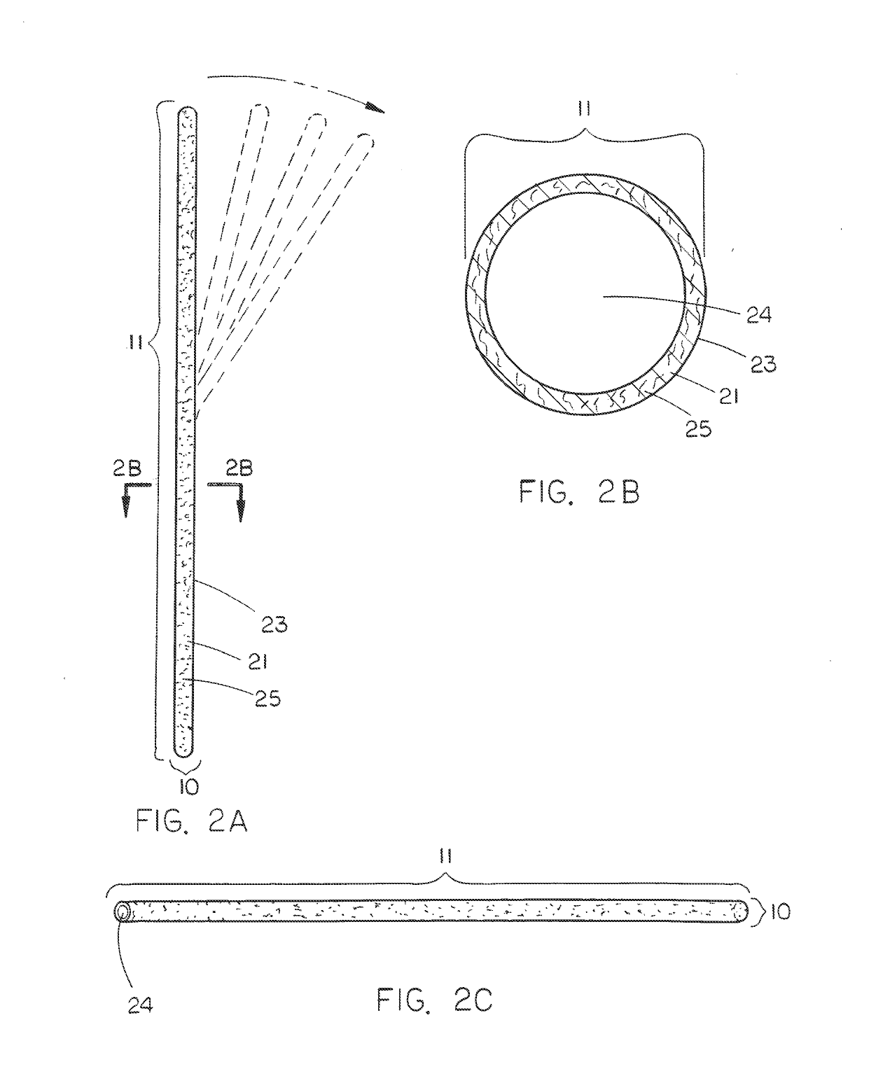

Looking now to FIG. 2A, in one embodiment, all or substantially all of the endotracheal intubation guide 10 is formed from a single material 21 that forms both the plastic element 25 of the endotracheal intubation guide 10 and all or substantially all of the body 11. The single material 21 can be any soft biocompatible material with sufficient plasticity or any blend of a variety of soft, biocompatible materials. Any variety of synthetic polymers, with the addition of appropriate plasticizers and co-polymers could form the single material 21. Polymers like polyethylene or polydiaxanone, for example, with the addition of one or more biocompatible plasticizers, such as polyethylene glycol, would have plasticity and malleability sufficient to practice the invention. Moreover, the addition of any variety of co-polymers would provide wide latitude to vary the plasticity, elasticity, give and durability of the single material 21 and, ultimately, the endotracheal intubation guide 10.

In embodiments where the single material 21 is comprised primarily of synthetic polymers, the single material 21 could be formed of woven polymers, extruded polymers, extruded cellular compositions of the synthetic polymers or as an extruded foam of the synthetic polymer. In a similar manner, the single the material 21 could also be formed of non synthetic polymers such as silk or rubber in order to obtain desired plasticity and flexibility.

The surface 23 of the endotracheal intubation guide 10 must gently engage the inner wall of the airway lumen 15, slidably engage the inner surface of an endotracheal tube and be able to traverse saliva, blood, mucus and other liquids that may obscure the patient's airway. In the present embodiment, the outer surface 23 of the endotracheal intubation guide 10 could be the interface of the single material 21 and the surface that the endotracheal intubation guide 10 is engaging. The single material 21 could be selected for, in addition to its characteristics enabling it to serve as the body 11 and the plastic element 25, for being low-friction and waterproof. In other embodiments outer surface 23 could constitute a thin coating of a distinct material. Forming the outer surface 23 of the endotracheal intubation guide 10 out of a material distinct from the single material 21 could be advantageous as the single material 21 could be exclusively selected for qualities specific to its operation as the body 11 and the plastic element 25.

Looking now to FIG. 2B and FIG. 2C, in another embodiment, the intubation guide is formed to contain a lumen 24 spanning all or substantially all of the body's length. The lumen 24 promotes ventilation during placement of the intubation guide. In one embodiment, the lumen 24 is of sufficient width so as to allow uninterrupted jet ventilation during placement of the endotracheal intubation guide 10.

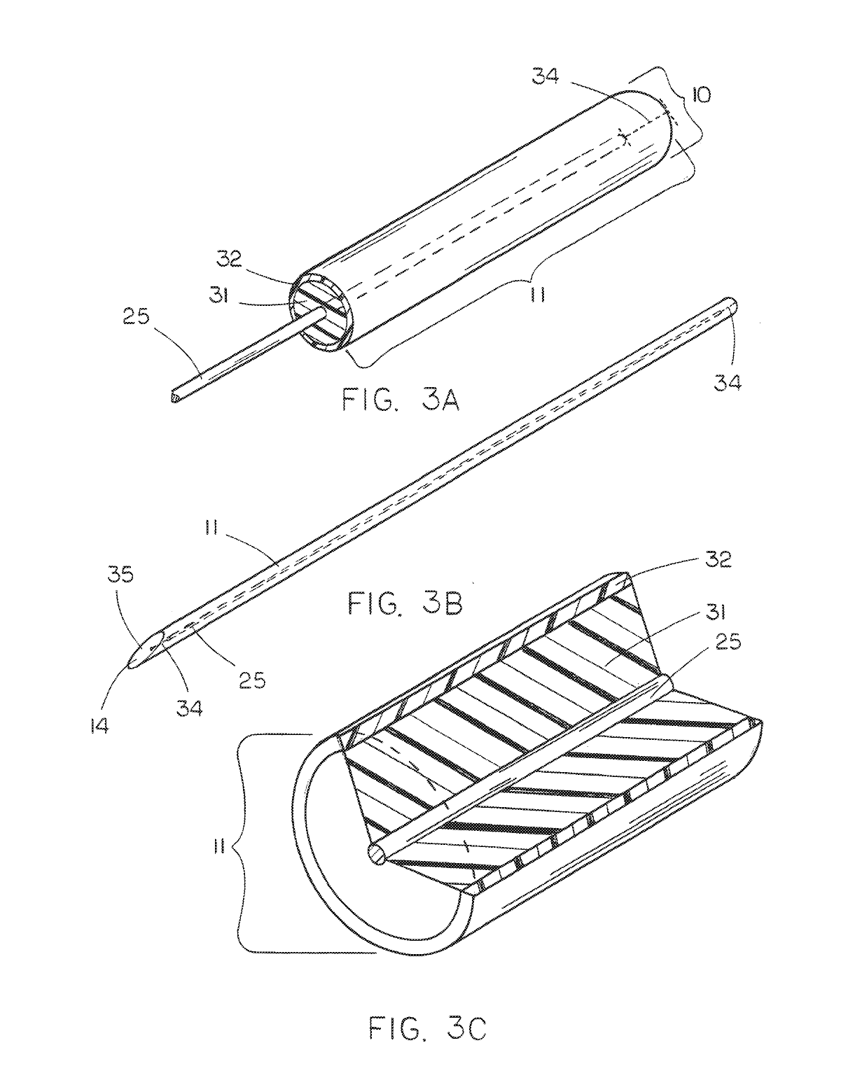

Looking now to FIG. 3A, in one embodiment the endotracheal intubation guide 10 is an assembly of more than one component. The endotracheal intubation guide 10 is formed substantially by a body 11. The body 11 is further formed to receive a plastic element 25. In the present embodiment, the plastic element 25 is a member formed from a malleable material sufficiently plastic to hold an angle bent into it. In a preferred embodiment, the plastic element 25 is a wire formed of a ductile metal such as steel, aluminum or copper. In other embodiments the plastic element 25 can be any similar flexible member that can undergo plastic deformation. The body 11 of the endotracheal intubation guide 10 is formed to completely surround the plastic element 25. So formed, the endotracheal intubation guide 10 can comprise a plastic element 25 that would, ordinarily, risk substantial trauma to the patient's airway. The body 11, formed of an appropriately soft material can entirely conceal the plastic element 25.

Looking at FIG. 3C, in a preferred embodiment, the body 11 is comprised of two components: an inner core 31, which is formed of a soft highly flexible material with minimal elasticity and a thin outer coat 32. The inner core 31 serves to protect the inner wall of the airway lumen 15 from the plastic element 25, to minimize the resistance against the plastic element 25 and maximize the plasticity of the endotracheal intubation guide 10. In a preferred embodiment the inner core 31 is formed of a biocompatible plasticized polyvinylchloride. Other embodiments can utilize similar biocompatible plasticized synthetic polymers in conjunction with other co-polymers. In other embodiments the inner core 31 can be formed of natural polymers such as silk or rubber. The inner core 31 could also be formed of polymerized, plasticized, biocompatible silicon oxides.

The outer coat 32 can be formed entirely by the interface of the inner core 31 and the surfaces engaged by the endotracheal intubation guide 10. The outer coat 32 can also be formed of a distinct material. For example, the outer coat 32 can be formed from a synthetic polymer optimized to be watertight and to have a low coefficient of friction such as a biocompatible thin layer of a plasticized polyvinylchloride resin. In other embodiments the outer coat 32 can be formed of thin layers of similar matter.

Looking now to FIG. 3B, to further protect the patient's airway from the plastic element 25, in preferred embodiments, the inner core 31 is formed to extend beyond the length of the plastic element 25 and create proximal and distal areas 34 comprised primarily of the inner core 31. Furthermore, the distal end 14 of the endotracheal intubation guide 10 can be formed to a different shape so as to facilitate engagement with the glottic opening 62 (not pictured). In a preferred embodiment the distal end 14 of the endotracheal intubation guide 10 is beveled 35 but in other embodiments the distal end 14 can be tapered, coude or otherwise shaped to facilitate engagement with the glottic opening 62 (not pictured).

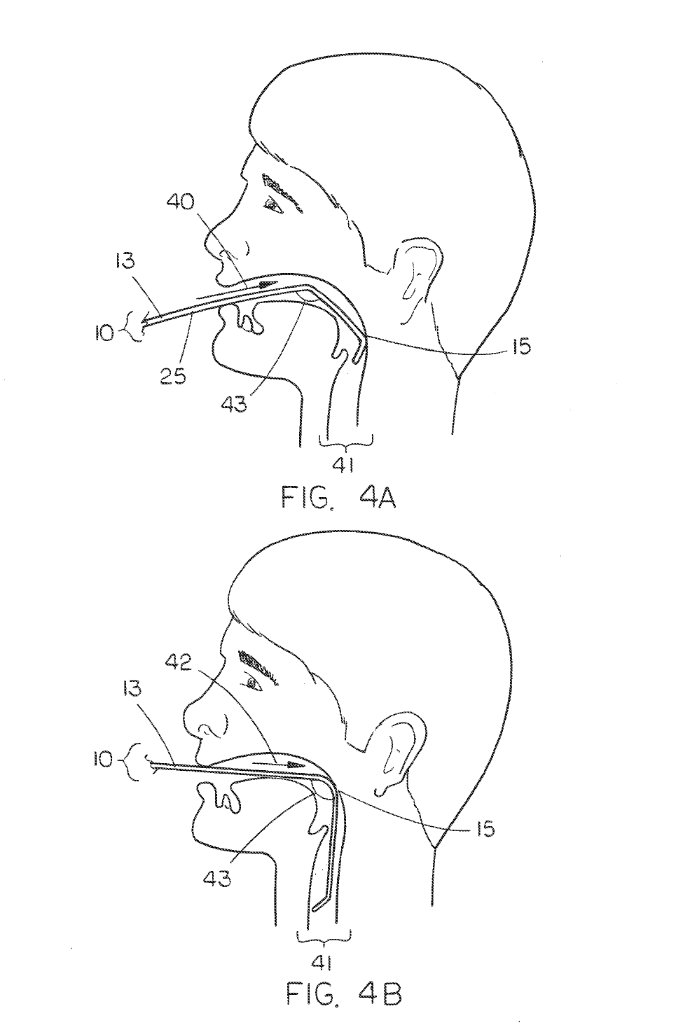

Critical to the practice of all embodiments of the invention is that the plastic element 25 must have sufficient give so as to minimize or prevent traumatic engagement with the inner surface of the airway lumen 15. Looking now to FIG. 4A, initial insertion 40 of an endotracheal intubation guide 10, shaped to substantially conform to the patient's endotracheal airway 41, is depicted. In all embodiments, the plastic element 25 of the endotracheal intubation guide 10 has sufficient give so that the engagement with the inner wall of the airway lumen 15 is non traumatic.

Looking now to FIG. 4B, the continued insertion 42 of the endotracheal intubation guide leads to the deformation of the endotracheal intubation guide 10 as it continues to engage the inner wall of the airway lumen 15. The deformation of the endotracheal intubation guide 10 when engaged with the inner surface of the airway lumen 15 constitutes non-traumatic engagement 43 with the inner surface of the airway lumen 15.

The give of the plastic element must be sufficient so that the force of initial insertion 40 or continued insertion 42 of the endotracheal intubation guide 10 will cause the endotracheal intubation guide 10 to deform when engaging the inner surface of the airway lumen 15 prior to traumatically engaging the inner surface of the airway lumen 15. For example, in embodiments where the plastic element 25 is a wire formed of ductile metal, the gauge of the wire must be thin enough to maximize the amount of give while still retaining plastic deformations so as to practice associated methods of indirect intubation. In preferred embodiments that utilize a steel wire, the plastic element 25 the gauge would be greater than 17 on the Washburn & Moen/U.S. Steel wire gauge scale.

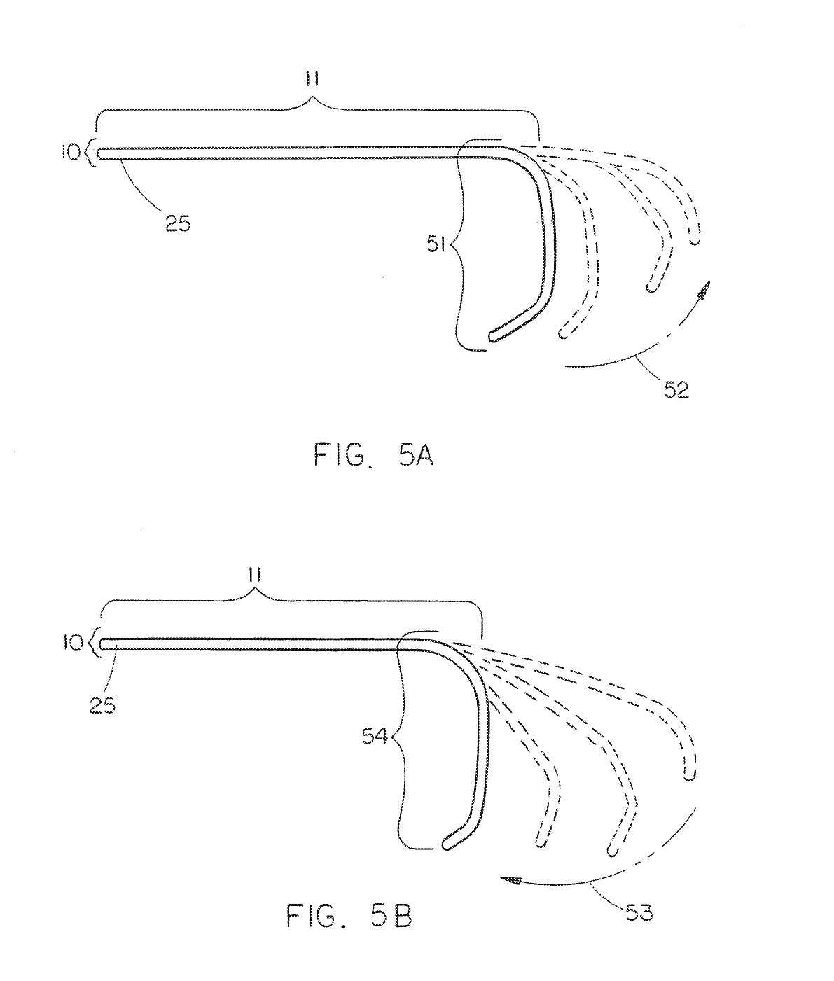

The present invention further advances the art by granting inexperienced intubators the option of using a pre-formed intubation guide. When presented with a linear, unbent endotracheal intubation guide (e.g. 10 as depicted in FIG. 1a) that is linear and contains no bends, an inexperienced intubator may not know how to shape the intubation guide so as to navigate the patient's airway (e.g. the shaped endotracheal intubation guide 13). FIG. 5A depicts embodiments of the endotracheal intubation guide 10 that are pre-formed to substantially conform to the unaligned shape of the patient's endotracheal airway 41 (not pictured). Moreover, the endotracheal intubation guide 10 can undergo elastic plastic deformation 52 that will return to the preformed shape of the endotracheal intubation guide or a shape that is substantially similar. In one embodiment, the endotracheal intubation guide 10 comprises a soft, malleable and minimally-resistant body 11 and a plastic element 25 that are able to permit elastic-plastic deformations 52. In one embodiment the plastic element 25 could be a member formed from an aluminum alloy selected for its ability to undergo elastic-plastic deformations. In other embodiments the plastic element 25 could be formed in part by the material substantially comprising the body 11 of the endotracheal intubation guide 10.

FIG. 5B depicts the endotracheal intubation guide 10, having undergone elastic-plastic deformation 52 returning 53 to a deformed shape 54 the angles comprising the endotracheal intubation guide 10 are biased towards the preformed shape 51 but are not exactly the same. In a preferred embodiment the pre-formed angles are maintained by a preformed plastic element 25 that can undergo elastic-plastic deformation 53. Depending on the skill of the intubator, and the intubator's associated experience using the endotracheal intubation guide 10, the degree of elasticity is variable. For inexperienced intubators, the amount of elasticity would be relatively high so that the plastic element 25 will bias deformations 52 towards the pre-formed shape 51 of the endotracheal intubation guide 10. Other embodiments would comprise a plastic element 25 that is less elastic so that experienced intubators could more readily deform and shape the endotracheal intubation guide 10. In some embodiments the plastic element 25 would have minimal or no elasticity so as to enable the intubator to shape the endotracheal intubation guide 10 to fit the patient's airway according to his or her judgment.

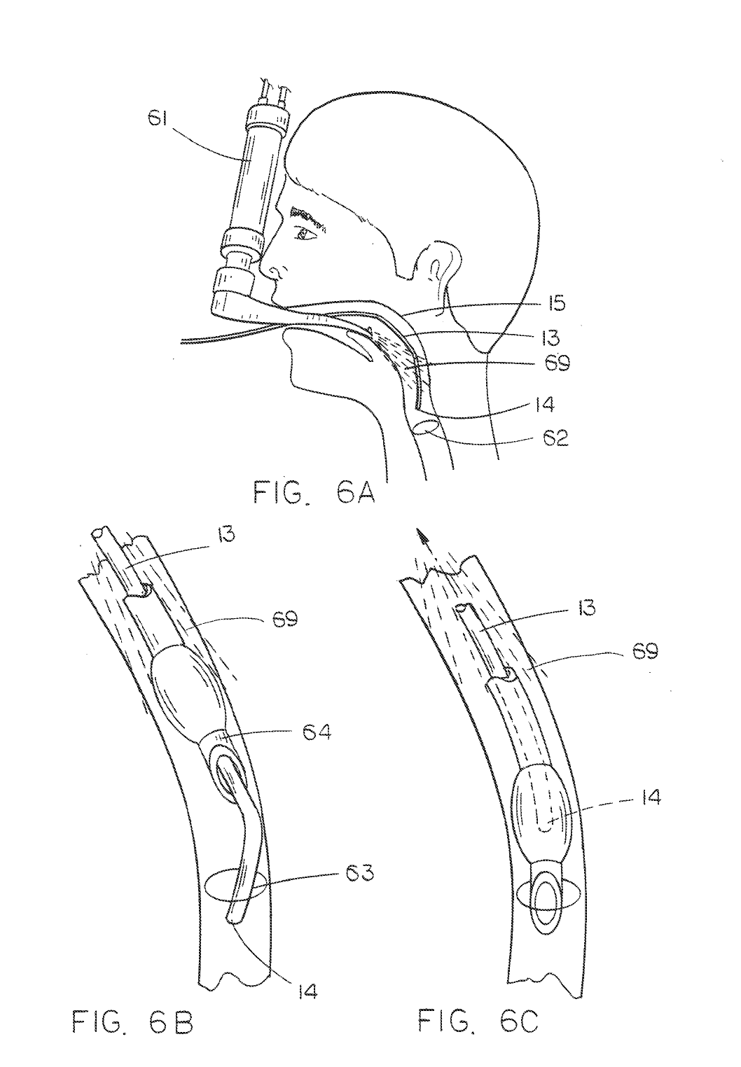

Looking now to FIG. 6A, in another embodiment, the present invention is a method for positioning devices in the patient's airway utilizing an indirect visualization instrument 61. In a preferred embodiment, the indirect visualization instrument is a Stortz video laryngoscope. The method involves obtaining a view of the glottic opening 62 utilizing the indirect visualization mode to see around anatomic obstructions. The intubator then places the shaped endotracheal intubation guide 13 so as to enable the placement of the distal end 14 around anatomical obstructions and into the field of view indirect visualization instrument 69. In another embodiment, the shaped endotracheal intubation guide 13 is pre-formed to substantially conform to the general shape of the endotracheal airway.

Critical to the present embodiment is that the intubator need not have the patient's airway 41 (not pictured) aligned so as to enable direct visualization of the glottic opening 62 (not pictured) using a indirect visualization instrument. The present art teaches that the first step of intubation is alignment of the patient's airway. Although the definition of a difficult airway is outcome-based, the factors influencing that outcome are often difficulties associated with anatomical obstructions to the alignment of the patient's airway. The practice of the present invention allows for successful intubation even when the patient's airway can be less than optimally aligned or cannot be aligned at all.

Looking to FIG. 6B and FIG. 6C, once the distal end 14 of the shaped endotracheal intubation guide 10 is in the field of view 69 of the indirect visualization instrument 61, the intubator can then confirm placement of the intubation guide 63 by indirect visualization. Upon confirmation of placement 63, the intubator can then slide an endotracheal tube 64 over the intubation guide. By maintaining the position of the indirect visualization instrument the intubator can subsequently confirm the placement of the endotracheal tube 64 prior to removing the shaped endotracheal intubation guide 13. Critically, placement of both the shaped endotracheal intubation guide 13 and the endotracheal tube 64 can be performed with minimal alignment of the patient's airway or even in an unaligned airway 17.

In other embodiments the method can utilize other means of indirect visualization. Other video laryngoscopes, such as the glidescope, can be used to indirectly visualize the glottic opening 62. Alternatively, a video laryngeal mask could be used to visualize the glottic opening 62. Even devices such as video stylets or fiber optic bougies could be used as indirect visualization instruments to confirm placement of the intubation guide and endotracheal tube. In preferred embodiments, however, the indirect visualization instrument provides a high-quality image and an abundant light source.

While the present invention has been described with reference to specific embodiments, it should be understood by those skilled in the art that varied changes may be made and equivalents may be substituted within the original scope of the invention and without changing the core of the invention. In addition, many modifications may be made to adapt to a particular situation. The present invention teaches many of the tools to make such modifications. All such modifications are intended to be within the scope of the present invention.

What is claimed is:

1. An apparatus to guide the placement of an endotracheal tube comprising:
(a) A non-rigid body formed to traverse an endotracheal tube's lumen and formed of a soft, minimally-resistant material;
(b) A plastic element that is able to retain plastic deformations when placed in minimal engagement with the inner surface of the airway lumen but with sufficient give so as to minimize traumatic engagement with the inner surface of the airway lumen.

2. The apparatus of claim 1 where the plastic element is received by the non-rigid body.

3. The apparatus of claim 1 where said body is formed of a member selected from a group comprising: latex, silicon, polyester, nylon, rubber, silk, or similar biocompatible material.

4. The apparatus of claim 1 where said body is formed from a synthetic polymer selected for biocompatibility and further comprising one or more plasticizers to promote plasticity.

5. The apparatus of claim 1 where said plastic element is a wire formed of a material selected from a group comprising: steel, copper, aluminum or a similarly ductile metal.

6. The apparatus of claim 1 where the surface of the non-rigid body is waterproof and promotes slidable engagement with an endotracheal tube.

7. The apparatus of claim 1 where the distal end is formed to promote insertive engagement with the glottic opening.

8. Apparatus of claim 1 where the body is formed to contain a lumen spanning all or substantially all of the body of the apparatus.

9. The apparatus of claim 1 where the apparatus is shaped to substantially conform to the lumen of an endotracheal airway and where the plastic element has sufficient elasticity so as to bias any deformation of the apparatus to substantially return to said formed shape that substantially conforms to the lumen of an endotracheal airway.

10. A method for endotracheal intubation comprising the steps of:
(a) Utilizing an indirect visualization instrument to visualize the airway
(b) Placing a series of plastic deformations into an intubation guide so as to place the distal end of the apparatus into the view of the indirect visualization instrument
(c) Utilizing the indirect visualization instrument to confirm the positioning of the intubation guide in the glottic opening
(d) Sliding an endotracheal tube over the intubation guide and position the endotracheal tube in the glottic opening
(e) Confirming the placement of the endotracheal tube with the indirect visualization instrument
(f) Withdrawing the intubation guide through the lumen of the endotracheal tube

11. The method of claim 10 where the indirect visualization instrument is selected from a group comprising: a Stortz DCI Video Laryngoscope or a Glidescope Video Laryngoscope, a generic video laryngoscope, a generic video laryngeal masks or a generic fiber optic bougie

12. The method of claim 10 where the airway is not aligned.
